(19) United States
(12) Patent Application Publication
(10) Pub. No.: US 2024/0417782 A1
(43) Pub. Date: Dec. 19, 2024

(54) METHOD AND KIT FOR GENETIC ANALYSIS

(71) Applicant: Hitachi, Ltd., Tokyo (JP)

(72) Inventors: Junko TANAKA, Tokyo (JP); Tatsuo NAKAGAWA, Tokyo (JP)

(21) Appl. No.: 18/582,764

(22) Filed: Feb. 21, 2024

(30) Foreign Application Priority Data
Jun. 15, 2023 (JP) 2023-098187

(51) Int. Cl. C12Q 1/6827; C12Q 1/6806; C12Q 1/686; C12Q 1/6876; G16B 20/20; G16B 20/50

(52) U.S. Cl. CPC C12Q 1/6827; C12Q 1/6806; C12Q 1/686; C12Q 1/6876; G16B 20/20; G16B 20/50

(57) ABSTRACT

A method and kit for genetic analysis can accurately determine whether a plurality of genetic mutations in close proximity are present in a positional relationship of a cis-form or a trans-form and quantify the genetic mutations. The method for genetic analysis includes: preparing probes corresponding to each of a plurality of genetic mutations; performing an amplification reaction using a solution comprising a primer for amplifying a region comprising the plurality of genetic mutations, the probes, a test biological sample, and an enzyme; measuring a binding of an amplicon to the probe by melting curve analysis at varying temperatures of the solution after performing the amplification reaction; and determining, based on a result of the melting curve analysis, whether the plurality of genetic mutations are on a same allele in a cis form or on different alleles in a trans form in DNA contained in the test biological sample.

### CROSS REFERENCE TO RELATED APPLICATIONS

The present application claims priority from Japanese patent application JP 2023-098187 filed on Jun. 15, 2023, the entire content of which is hereby incorporated by reference into this application.

### SEQUENCE LISTING

The present application contains a Sequence Listing which has been submitted electronically in XML format and is hereby incorporated by reference in its entirety. Said XML copy, created on May 24, 2024, is named H_A-14610_SL.xml and is 10,715 bytes in size.

### BACKGROUND

### Technical Field

The present invention relates to a method and kit for genetic analysis for analyzing a plurality of genetic mutations, specifically, a method and kit for genetic analysis for analyzing a plurality of genetic mutations using a probe for melting curve analysis. The present invention also relates to a methods and kit for assessing a trait (e.g. drug responsiveness) associated with a plurality of genetic mutations.

### Background Art

Non-small-cell lung cancer (NSCLC) accounts for 80 to 90% of all lung cancers, and genetic mutations in epidermal growth factor receptor (EGFR) and other driver genes are frequently observed, for which molecular target drugs have been developed. EGFR tyrosine kinase inhibitors (EGFR-TKIs), which are molecular target drugs for EGFR genetic mutations, include gefitinib and erlotinib as the first generation drugs, and afatinib and dacomitinib as the second generation drugs. However, patients with the EGFR genetic mutations who respond to these EGFR-TKIs develop a resistance mutation, T790M, in the first year or so after the start of treatment. Therefore, osimertinib has been developed as a third generation EGFR-TKI that is also effective in patients with the T790M mutation. However, the lack of response to osimertinib due to the occurrence of C797S as a resistance mutation of osimertinib has become a new problem.

In recent years, it has been found that the positional relationship between the T790M and C797S genetic mutations can alter the therapeutic efficacy (Uchibori, et al., Nat. Commun., 8, 14768, 2017). When T790M and C797S are present in a trans form (on different alleles), existing EGFR-TKIs can be used in combination to provide treatment successfully because osimertinib is effective for T790M while first and second generation EGFR-TKIs are effective for C797S. On the other hand, when T790M and C797S are present in a cis form (on a same allele), it has been found that all EGFR-TKIs, including the combination of existing EGFR-TKIs, are ineffective. A cis-trans determination method of genetic mutations is important because it is possible to develop an effective therapeutic drug or decide whether to administer the therapeutic drug by determining the positional relationship between the mutations involved in the sensitivity and resistance to the treatment.

The cis-trans determination method for T790M and C797S have been reported as follows: first, hydrolysis probes corresponding to each of T790M and C797S, which are labeled with different fluorescent dyes, are prepared. DNA polymerase, primers, probes, and enzymes required for PCR are added to a sample to perform digital PCR. In the digital PCR, the reaction solution is divided into micro partitions, such as wells or droplets, and amplified in the micro partitions. Since in a trans-form sample, T790M and C797S are present in different genes, one micro partition contains only one genetic mutation with only one fluorescence being detected. Since in a cis-form sample, T790M and C797S are present in the same gene, one micro partition contains both genes with two types of fluorescence being detected. Then, the number of micro partitions with only one fluorescence being detected and the number of micro partitions with two types of fluorescence being detected can be each counted to determine whether the genetic mutations in the sample are in a cis form or in a trans form.

### SUMMARY

In digital PCR using hydrolysis probes labeled with two fluorescent dyes, however, a large number of copies result in a high probability of simultaneous entry of DNA with T790M and C797S in trans-form into a single micro partition during dispensation, leading to erroneous detection as DNA in a cis form. In addition, due to high probability of the simultaneous entry of the DNA with T790M and C797S in trans-form into a single micro partition, it is difficult to accurately quantify the ratio of cis-and trans forms when they are mixed together.

Therefore, an object of the present invention is to provide a method and kit for DNA detection that can accurately determine whether multiple genetic mutations in close proximity are present in a positional relationship of a cis-form or a trans-form and quantify the genetic mutations.

The inventors had developed a technique for combining digital PCR with melting curve analysis by using a non-degradable probe such as a molecular beacon instead of a hydrolysis probe to identify the genotype of a target gene with high sensitivity and high multiplexing by melting curve analysis after amplification (US2019/0352699A1 and Nakagawa, et al., Anal. Chem., 92, 11705-11713, 2020). The present inventors have found that when molecular beacons designed corresponding multiple genetic mutations in close proximity are prepared and subjected to melting curve analysis, the shape of the melting curve changed between the cis and trans forms due to steric hindrance between the molecular beacons, resulting in a different melting temperature (Tm) calculated from the melting curve. Based on the differences in melting curves and melting temperatures, we have found that the cis and trans forms of a plurality of genetic mutations can be determined and quantified, thus completing the present invention.

In one aspect, the present invention provides a method for genetic analysis comprising the steps of:
preparing probes corresponding to each of a plurality of genetic mutations;
performing an amplification reaction using a solution comprising a primer for amplifying a region comprising the plurality of genetic mutations, the probes, a test biological sample, and an enzyme;
measuring a binding of an amplicon to the probe by melting curve analysis at varying temperatures of the solution after performing the amplification reaction; and
determining, based on a result of the melting curve analysis, whether the plurality of genetic mutations are on a same allele in a cis form or on different alleles in a trans form in DNA contained in the test biological sample.

In another aspect, the present invention provides:
a method for conducting genetic analysis on a test biological sample from a subject to assess a trait associated with a plurality of genetic mutations, the method comprising the steps of;
performing the method above using the test biological sample from a subject to determine whether the plurality of genetic mutations are on a same allele in a cis form or on different alleles in a trans form in the test biological sample from the subject; and
assessing whether the subject has the trait associated with the plurality of genetic mutations.

In a further aspect, the present invention provides a kit for genetic analysis comprising:
a primer pair for amplifying a region comprising a plurality of genetic mutations, the primer pair comprising a forward primer and a reverse primer; and
a plurality of probes for melting curve analysis, each corresponding to the plurality of genetic mutations, which bind to a product amplified using the forward primer and the reverse primer,
wherein the probes have different melting curve characteristics or melting temperatures when the plurality of genetic mutations are present on a same allele and on different alleles.

The present invention provides a method and kit for genetic analysis that can more accurately and sensitively determine whether multiple genetic mutations in close proximity are in cis or trans forms as the positional relationship or quantify the genetic mutations. Accordingly, the present invention may be useful in fields of, for example, basic research, testing, and drug discovery for gene detection, genotyping, and the like.

### BRIEF DESCRIPTION OF THE DRAWINGS

FIGS. 1A to 1C are schematic diagrams showing an example of a method of measuring a melting temperature of DNA using a fluorescent-labeled probe;

FIGS. 2A and 2B are schematic diagrams showing an example of a method of measuring melting temperatures of DNAs with genetic mutations in a trans form shown in

FIG. 2A and in a cis form shown in FIG. 2B using a fluorescent-labeled probe according to an embodiment of the present invention;

FIGS. 3A to 3F illustrate an example of melting curves when mutations in a trans form (FIGS. 3A and 3B) and in a cis form (FIGS. 3C to 3F) are measured using a fluorescent-labeled probe according to one embodiment of the present invention;

FIG. 4 illustrates an example of a first database that stores temperatures for each genetic mutation in a cis form and in a trans form according to one embodiment of the present invention;

FIG. 5 is a flowchart showing one embodiment of a method for genetic analysis to determine whether genetic mutations in a biological sample are in a cis form or in a trans from using melting curve analysis;

FIG. 6 is a flowchart showing one embodiment of a method for genetic analysis using melting curve analysis in combination with digital PCR to quantify the number of mutant copies of a gene and a ratio of cis to trans forms of genetic mutations;

FIGS. 7A to 7D show measurement results when genes with T790M mutation only, C797S mutation only, the mutations in a cis form, and in a trans form are amplified by real-time PCR to conduct melting curve analysis in the Examples of the present invention; and

FIGS. 8A to 8H show measurement results when genes with T790M mutation only, C797S mutation only, the mutations in a cis form, and in a trans form are amplified by digital PCR to conduct melting curve analysis in the Examples of the present invention.

### DETAILED DESCRIPTION

The object, features, and advantages of the present invention, and ideas related thereto will be apparent to those skilled in the art from the description of the present specification. From the description of this specification, those skilled in the art can easily reproduce the present invention. Embodiments and specific Examples of the invention described below are intended to illustrate preferred embodiments of the invention and provided for illustrative or descriptive purposes, which are not intended to limit the invention thereto. It will be apparent to those skilled in the art that various changes and modifications can be made according to the description of in the present invention without departing from the spirit or scope of the present invention disclosed herein.

### (1) Method for Genetic Analysis

In one aspect, the present invention provides a method for genetic analysis, the method comprising the steps of:
preparing probes corresponding to each of a plurality of genetic mutations;
performing an amplification reaction (e.g., PCR) using a solution comprising a primer for amplifying a region comprising the plurality of genetic mutations, the probes, a test biological sample, and an enzyme;
measuring a binding of an amplicon to the probe by melting curve analysis at varying temperatures of the solution after performing the amplification reaction; and determining, based on a result of the melting curve analysis, whether the plurality of genetic mutations are on a same allele (in a cis form) or on different alleles (in a trans form) in DNA contained in the test biological sample.

As used herein, genetic analysis means, for example, determining whether a plurality of genetic mutations to be analyzed are present in a test biological sample, determining whether a plurality of genetic mutations are on a same allele (in a cis form) or on different alleles (in a trans form), measuring an abundance ratio or the abundance of cis and trans forms of a plurality of genetic mutations to be analyzed, and monitoring the presence of cis and trans forms of a plurality of genetic mutations.

According to the present invention, the test biological sample may not be particularly limited as long as it is a biological sample that contains DNA containing or possibly containing a genetic mutation to be analyzed. For example, the biological sample may include a body fluid sample such as blood or urine, a tissue, a cultured cell, DNA extracted and purified from these samples (genomic DNA, circulating DNA, etc.), or DNA derived from mRNA or the like extracted and purified from the samples (cDNA, etc.). Alternatively, synthesized DNA may be used as a test biological sample for the design of a suitable probe or primer, for example. When the test biological sample is, for example, a body fluid sample, a tissue, and a cultured cell, it may be preferable to treat the sample in advance to make DNA or the like available for the amplification reaction performed in the method of the present invention. Methods for preparing DNA from such samples are known in the art, and kits for conveniently purifying DNA and for extracting mRNA to synthesize cDNA are also commercially available.

The genetic mutations to be analyzed according to the present invention may be a plurality of genetic mutations for which it is desired to determine whether they are on a same allele (in a cis form) or on different alleles (in a trans form). The expression “on a same allele (in a cis form)” means that a plurality of genetic mutations are present on a same allele of two alleles in a chromosome, and the expression “on different alleles (in a trans form)” means that a plurality of genetic mutations are present on different alleles of two alleles in a chromosome. Since it may be desirable to conduct melting curve analysis on a plurality of genetic mutations with each probe rather than with a common single probe, the mutations may be separated by 5 bases or more. In addition, the distance between the mutations may be shorter than 50 bases so that a change in the melting curve due to steric hindrance between the probes occurs. Therefore, the plurality of genetic mutations may be separated by, for example, 5 to 60 bases, preferably 5 to 40 bases. However, genetic mutations can be more distant from each other if a change in the melting curve occurs due to steric hindrance between probes.

Specific examples of the genetic mutations to be analyzed may include, but are not limited to, T790M and C797S in the epidermal growth factor receptor (EGFR) gene, T790M and L858R in the EGFR gene, exon 19 deletion mutations (active mutations) in the EGFR gene, any combination of R88, R108, E453, E542, E545, E726 and H1047R mutations in the PIK3CA gene, and resistant mutations C1156Y, 1117 IN, 11171S, 11171T, F1174C, V1180L, L1196M, G1202R, G1202 deletion, D1203N, E1210K and G1269A in the ALK gene.

In the method of the present invention, probes corresponding to each of a plurality of genetic mutations may be prepared. The probe may be designed so that at least a part of the probe has a sequence specific to a nucleotide sequence of the genetic mutations to be analyzed, that is, a sequence complementary to the nucleotide sequence of the genetic mutations. Since the probe is required for each of a plurality of genetic mutations, a number of probes corresponding to the types of the genetic mutations to be analyzed may be prepared. Probe design techniques are well known in the art, and probes that can be used according to the present invention may be designed to have a length and base composition (melting temperature) that allow for specific binding (hybridization). For example, the length of the probe which performs its function may be preferably 10 bases or more, more preferably 15 to 50 bases, and even more preferably 15 to 30 bases, for example, about 20 bases, as the base length of a sequence portion specific to a genetic mutation. It may be preferable to confirm the GC content of the probe and the melting temperature (Tm) of the probe in designing. For the confirmation of Tm, a known software for designing a probe can be used.

According to the present invention, the terminals of each probe may be closer than 20 bases to each other so that the change in the melting curve due to steric hindrance between the probes for each of a plurality of genetic mutations occurs. Although an overlapping region between the terminals of the probes may cause a decrease in the binding amount, the overlapping region between the terminals of the probes may be acceptable because the probes can be detected in the cases when the probes have high affinity, or the fluorescent dye of the probe has high fluorescent intensity, a detector with high fluorescent detection sensitivity is used. It may be desirable that the terminals of the probes may be separated by 2 bases or more, or even 4 bases or more. When the terminals of the probes are separated by 10 bases or more, the amount of change in the melting curve due to steric hindrance can be adjusted by inserting a linker between the nucleotide sequence of the probe and the fluorescent dye or changing the size of the fluorescent dye.

The probe may comprise a fluorescent dye or may comprise a fluorescent dye and a quenching dye. When the probe comprises a fluorescent dye, the fluorescent dye may be used to measure a binding of an amplicon to the probe. In one embodiment, the 3′- and 5′-terminal sequences of the probe may have complementary sequence portions, and the 3′- and 5′-terminal sequences of the probe may bind in a free state (when not bound to the amplicon) to form a stem-loop structure. In other words, the probe may preferably be a molecular beacon.

Designed probes can be chemically synthesized by known oligonucleotide synthesis methods but are usually synthesized using commercially available chemical synthesis apparatus.

Subsequently, an amplification reaction may be performed. The amplification reaction may be performed on a solution containing a primer for amplifying a region comprising the plurality of genetic mutations, the probes, a test biological sample, and an enzyme.

Primer design techniques are also well known in the art, and a primer that can be used according to the present invention may be designed to meet conditions for specific annealing, for example, to have a length and base composition (melting temperature) that allow for specific annealing. For example, the length of the primer which performs its function may be preferably 10 bases or more, more preferably 15 to 50 bases, and even more preferably 15 to 30 bases, for example, about 20 bases. It may be preferable to confirm the GC content of the primer and the melting temperature (Tm) of the primer in designing. For the confirmation of Tm, a known software for designing a primer can be used. Designed primers can be chemically synthesized by known oligonucleotide synthesis methods but are usually synthesized using commercially available chemical synthesis apparatus.

The enzyme used for the amplification reaction may be a polymerase capable of performing an extension reaction using DNA as a template, and any known polymerase can be used.

The amplification step can be performed by any amplification reaction known in the art. Preferably, the amplification step may be performed by asymmetric PCR. In this case, the amount of either a forward primer or a reverse primer added may be increased to perform the amplification reaction. For the asymmetric PCR, see, for example, the description in Anal. Chem., 92, 11705-11713, 2020 (Nakagawa, et al., Anal. Chem., 92, 11705-11713, 2020).

In one embodiment, the binding of an amplicon to the probe may be measured by melting curve analysis at varying temperatures of the solution. Since the binding may vary depending on the melting temperature, the melting temperature of a double strand between the amplicon and the probe can be calculated from the change in binding due to the temperature change of the solution. Alternatively, the binding of the amplicon to the probe may be measured for each cycle of the change in the temperature of the solution, and the target genetic mutation can be determined from the relationship between the number of cycles and the change in binding.

Through the amplification reaction and melting curve analysis as described above, it becomes possible to determine whether a plurality of genetic mutations are present in a cis form or in a trans form for the DNA in the test biological sample based on the difference in the characteristics of the melting temperature or melting curve between those in case of that the plurality of genetic mutations are present in a cis form and those in case of that the plurality of genetic mutations are present in a trans form.

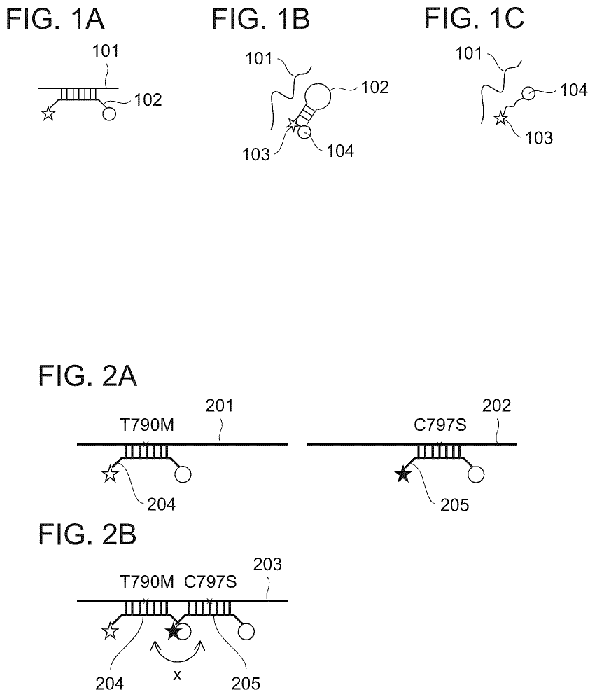

The method for genetic analysis of the present invention will be described in detail with reference to schematic diagrams of FIG. 1A to FIG. 6.

FIGS. 1A to 1C are schematic diagrams showing an example of a method of measuring a melting temperature of DNA using a fluorescent-labeled probe, for example, a molecular beacon or a Pleiades probe. Here, FIGS. 1A-1C are described using a molecular beacon as an example.

A fluorescent-labeled probe 102, which is a molecular beacon, is prepared as an oligonucleotide, and has a sequence complementary to a sequence (amplicon) between a pair of primers used in an amplification reaction for amplifying a target gene. The molecular beacon also has a complementary sequence portion at both terminals, and a fluorescent dye 103 is provided at one end and a quenching dye (quencher) 104 is provided at the other end. In the initial state (free state) of the amplification reaction, the molecular beacon 102 may exist alone and free, as shown in FIG. 1B. At that time, the molecular beacon 102 may form a stem-loop at the complementary sequence portion, and the fluorescent dye 103 and the quencher 104 are close to each other, resulting in no emission of fluorescence. When the sample solution is heated in the first denaturation step, it takes on a structure with a high degree of freedom, as shown in FIG. 1C. However, since the fluorescent dye and the quenching dye ordinarily do not separate, the fluorescence may remain quenched. When the temperature is lowered to about room temperature in an annealing step, a loop portion of the molecular beacon 102 may anneal to DNA 101 amplified in the sample solution, as shown in FIG. 1A, causing the fluorescent dye 103 and the quencher 104 to always separate. Thus, the fluorescent-labeled probe 102 may emit strong fluorescence. In the next extension step, the molecular beacon 102 may be released and may become again as shown in FIG. 1B, and the fluorescence may be quenched. In the next denaturation step, the molecular beacon 102 may become again as shown in FIG. 1C, and the fluorescence may remain quenched. Since in the amplification reaction, the above process is repeated, the fluorescent intensity can be measured at some point during heating or cooling. The fluorescent intensity can be measured in the same manner after completion of the amplification reaction. After completion of the amplification reaction, the fluorescence intensity may be detected with heating or cooling just to measure the fluorescence intensity. A melting curve may be created by plotting a change in the fluorescent intensity with a change in the temperature at this time to calculate the melting temperature from the melting curve.

In the molecular beacon 102 used herein, the combination of the fluorescent dye 103 and the quencher 104 may not be particularly limited as long as it is a combination that is generally used in real-time PCR. Examples of the fluorescent dye 103 may include FAM, VIC, ROX, Cy3, and Cy5, and examples of the quencher 104 may include TAMRA, BHQ1, BHQ2, and BHQ3, all of which are used conventionally and are available as commercial products.

When two genetic mutations with different sequences are to be analyzed, the two target genetic mutations can be differentially detected in one reaction system by preparing the sequence of the molecular beacon 102 that specifically binds to each of the target genetic mutations, and binding different fluorescent dyes thereto. Alternatively, the two target genetic mutations can be differentially detected in one reaction system even when the same fluorescent dyes are bound by designing the sequence of the molecular beacon such that the melting temperatures of the two molecular beacons are different.

FIGS. 2A and 2B are schematic diagrams showing an example of a method of measuring melting temperatures of DNAs with genetic mutations in a trans form and in a cis form using a fluorescent-labeled probe according to an embodiment of the present invention. In FIGS. 2A and 2B, the T790M and C797S mutations in the EGFR gene are shown as examples of genetic mutations. T790M and C797S are genetic mutations that occur 19 bases apart, and fluorescent-labeled probes (e.g., molecular beacons) may be prepared for each mutation. When molecular beacons are used as fluorescent-labeled probes, the molecular beacons may be designed to have specific sequences in their loops such that the mutation of T790M or C797S is close to the center, and the sequence of the loops may be 10 to 30 bases. As shown in FIG. 2A, since T790M and C797S are on separate allele in the trans-form gene, only fluorescent-labeled probes (molecular beacon) 204 corresponding to T790M may bind to DNA 201 containing the T790M mutation in a trans form, and only fluorescent-labeled probe (molecular beacon) 205 corresponding to C797S may bind to DNA 202 containing the C797S mutation in a trans form. As shown in FIG. 2B, since T790M and C797S are on the same allele in DNA 203 in a cis-form, molecular beacon 204 for T790M and molecular beacon 205 for C797S may bind simultaneously. As a result, steric hindrance occurs between the molecular beacon 204 for T790M and the molecular beacon 205 for C797S in DNA 203 in a cis-form, which may reduce the ease of binding of the molecular beacons compared with DNAs 201 and 202 in a trans form.

When the fluorescent-labeled probes (molecular beacons) corresponding to T790M and C797S have low specificity and also weakly bind to their respective wild-types (T790 and C797), the fluorescent-labeled probes corresponding to T790M and C797S may also bind to the trans-form DNA and have a binding mode similar to that of the cis form. In that case, the probes (blockers) corresponding to the wild-type gene without any modification of the fluorescent dye or quencher may be used to inhibit nonspecific binding of the fluorescent-labeled probes and to avoid the steric hindrance on the trans-form DNA. Specifically, the fluorescent-labeled probes and blockers corresponding to T790M and C797S can bind to DNAs with T790M and C797S, respectively, without any steric hindrance. The probes (blockers) corresponding to the wild-type gene can be appropriately designed by those skilled in the art based on the nucleotide sequence of the wild-type gene. In one embodiment, therefore, in the step of performing the amplification reaction, the amplification reaction can be performed with an addition of an additional probe corresponding to the wild-type gene to the solution.

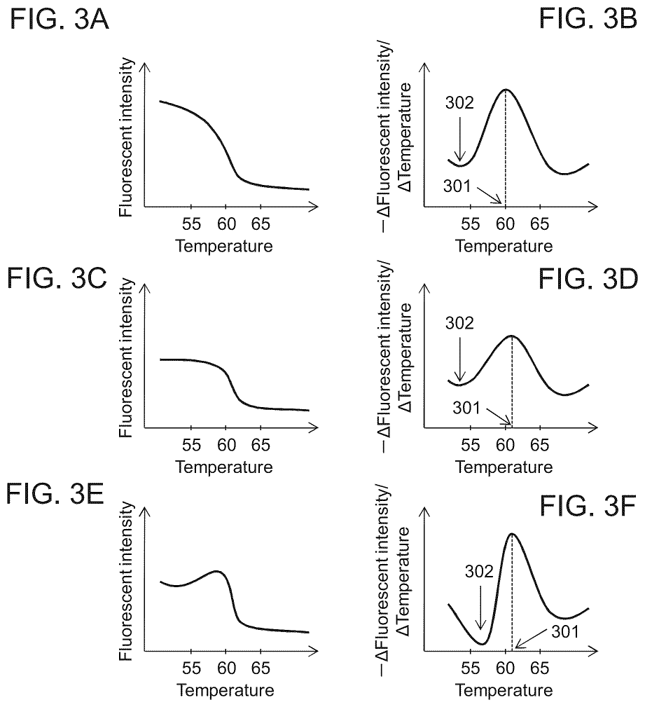

FIGS. 3A to 3F illustrate an example of melting curves when mutations in a trans form and in a cis form are measured using fluorescent-labeled probes. FIG. 3A shows a melting curve when mutations in a trans form are measured, while FIG. 3B shows a differential curve of the melting curve. As shown in FIG. 3A, the fluorescent-labeled probes may bind to the corresponding DNA at low temperature and show high fluorescent intensity. As the temperature increases, the fluorescent intensity may decrease due to the dissociation of the fluorescent-labeled probes from the corresponding DNA. When the differential curve is obtained from FIG. 3A, it shows a high peak at 60° C. as shown in FIG. 3B, and the temperature of the peak is a melting temperature (Tm) 301. On the other hand, FIG. 3C shows an example of the melting curve when mutations in a cis form are measured, while FIG. 3D shows a differential curve thereof. In the case of the cis form, the binding amount of the fluorescent-labeled probes may decrease due to the steric hindrance between the fluorescent-labeled probes corresponding to each mutation, resulting in a decrease in the fluorescent intensity at low temperature. After the fluorescent-labeled probes corresponding to one of the mutations dissociate, the steric hindrance may be eliminated, resulting in the same change in the fluorescent intensity as that of the trans form, as shown in FIG. 3C. When the differential curve is obtained, the height of the peak may decrease as shown in FIG. 3D, and the temperature (melting temperature) 301 of the peak may shift to the high temperature side. FIG. 3E shows another example of the melting curve when mutations in a cis form are measured, while FIG. 3F shows a differential curve thereof. In the example of FIG. 3E, the steric hindrance may be eliminated due to the dissociation of the fluorescent-labeled probes corresponding to one of the mutations around 55° C., resulting in an increase in the binding amount of the fluorescent-labeled probes corresponding to the other mutation, as well as an increase in the fluorescent intensity from 55° C. to 60° C. Therefore, in FIG. 3F, which is the differential curve of FIG. 3E, a large negative peak 302 occurs between 55° C. and 60° C. In addition, the melting temperature 301 of the peak shifts to the high temperature side.

Melting curve characteristics that can be used to discriminate between mutations in a trans form and in a cis-form may include the following: melting temperature; left-right balance of the width of the peak shape in the differential curve of the melting curve; magnitude of the local minimum on a low temperature side of the local maximum in the differential curve of the melting curve; slope(s) of left and right sides of the peak in the differential curve of the melting curve and a balance thereof (which can be calculated by second derivative); height of the local maximum and height of the local minimum in the differential curve of the melting curve; and ratio thereof. Any one or any combination of these characteristics can be used to determine whether a plurality of genetic mutations are present and, if present, whether they are in a trans form or in a cis form.

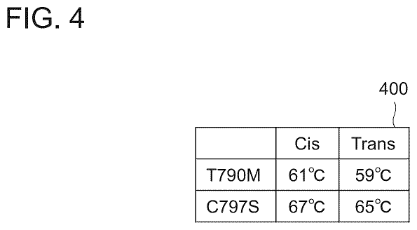

FIG. 4 illustrates an example of a first database that stores temperatures for each genetic mutation in a cis form and in a trans form. As illustrated in FIG. 3, melting temperatures may vary between the cis and trans forms. Therefore, the melting temperatures of the cis and trans forms with respect to T790M and C797S may be measured in advance using control DNA and stored in a database 400 as shown in FIG. 4. By measuring a biological sample for which the information on genetic mutations is unknown and as melting temperature thereof, it may be possible to determine whether the genetic mutations in the biological sample are in a cis form or in a trans form by comparing it with the melting temperature in the database 400. If the melting temperature of T790M in a trans form is observed and neither C797S in a trans form nor in a cis form is observed, it can be determined that only T790M is present and C797S is absent. In one embodiment, therefore, the melting temperature for the DNA contained in the test biological sample can be compared with the first database that stores melting temperature information corresponding to each of cis and trans forms of the plurality of genetic mutations to determine whether the plurality of genetic mutations are in a cis form or in a trans form. In this case, for example, the number of copies of the genetic mutations, the number of copies of genetic mutations in a cis form, and the number of copies of genetic mutations in a trans form may be each counted for the DNA contained in the test biological sample using the first database.

In FIG. 4, T790M and C797S are used as examples of genetic mutations to be stored in the database 400, but the genetic mutations may not be limited thereto or even two; there can be three or more.

An example of the information to be stored in the database may be an amount of characteristics of shapes of the melting curve and the differential curve thereof as shown in FIGS. 3A to 3F, and examples thereof may include a dip added to a sigmoid curve such as the melting curve in FIG. 3E and the magnitude of a negative peak such as the differential curve of the melting curve in FIG. 3F. In one embodiment, therefore, the melting curve for the DNA contained in the test biological sample can be compared with the second database that stores melting curve characteristics corresponding to each of cis and trans forms of the plurality of genetic mutations to determine whether the plurality of genetic mutations are in a cis form or in a trans form. Examples of the melting curve characteristics to be stored in such a second database may include, but are not limited to, at least one selected from the group consisting of a melting temperature, a left-right balance of a width of a peak shape in a differential curve of the melting curve, a magnitude of a local minimum on a low temperature side of a local maximum in a differential curve of the melting curve, a slope(s) of left and right sides of a peak in a differential curve of the melting curve and a balance thereof, a height of a local maximum and a height of a local minimum in a differential curve of the melting curve, and a ratio thereof. In this case, for example, the number of copies of the genetic mutations, the number of copies of genetic mutations in a cis form, and the number of copies of genetic mutations in a trans form may be each counted for the DNA contained in the test biological sample using the second database.

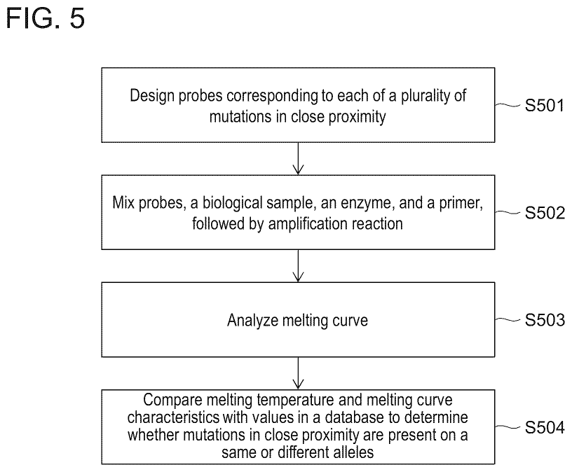

FIG. 5 is a flowchart showing one embodiment of a method for genetic analysis to determine whether genetic mutations in a biological sample are in a cis form or in a trans from using melting curve analysis. First, probes corresponding to each of multiple mutations in close proximity may be designed (S501). The probes may be mixed with a biological sample, an enzyme, and a primer to perform amplification reaction (PCR) (S502). The melting curve of the probe corresponding to each mutation and the amplified DNA may be obtained by melting curve analysis (S503). The melting temperature and melting curve characteristics may be compared with values in the database to determine whether mutations in close proximity are present on a same allele or different alleles (S504).

The amplification step by PCR can be performed by any amplification reaction known in the art. For example, it may be preferable to use a quantifiable amplification reaction as the amplification reaction. Real-time PCR and digital PCR, for example, can be used. Preferably, the amplification step may be performed by asymmetric PCR. In this case, the amount of either a forward primer or a reverse primer added may be increased to perform the amplification reaction. For the asymmetric PCR, see, for example, the description in Nakagawa, et al., Anal. Chem., 92, 11705-11713, 2020.

The binding of the amplicon to the probe may be measured with a change in the temperature. Since the binding may vary depending on the melting temperature, the melting temperature of a double strand between the amplicon and the probe can be calculated from the change in binding due to the temperature change.

The binding of the amplicon to the probe may be measured for each cycle of the change in the temperature, and the target mutation may be detectable from the relationship between the number of cycles and the change in binding. When the fluorescent dye of the probe is different for each mutation, the binding of the amplified product to the probe may be measured while changing a fluorescent filter for each cycle of the change in the temperature, while the ratio of the initial concentrations of a plurality of target mutations in the DNA of the test biological sample can be calculated from the relationship between the number of cycles and the change in binding.

The binding of the amplicon to the probe may be measured for each cycle of the change in the temperature, and the relationship between the number of cycles and the change in binding can be compared with the change in binding of a sample containing a known concentration of the target mutation to calculate the initial concentration of the target mutation with unknown concentration in the DNA of the test biological sample.

For example, a probe for T790M may be modified with the fluorescent dye FAM, and a probe for C797S may be modified with the fluorescent dye Cy5. Each probe may be mixed with a biological sample containing the DNA of the target genetic mutation, an enzyme, and a primer to perform real-time PCR. In the real-time PCR, the fluorescent intensity due to the binding of the probe to the amplicon may be measured while changing a fluorescent filter for each cycle of the change in the temperature. The relationship between the number of cycles and the fluorescent intensity can be compared with a change in the fluorescent intensity of the sample containing the target mutation at a known concentration to calculate the initial concentrations of T790M and C797S in the biological sample. After the real-time PCR, melting curve analysis may be performed to obtain a melting curve and its differential curve for each of FAM and Cy5, and a melting temperature calculated from the differential curve. It may be compared with the melting temperature obtained in advance using the control DNA which is stored in a database, and it may be determined whether T790M and C797S are in the positional relationship of a cis-form or a trans-form. If the melting temperature is an intermediate value between cis and trans forms, or the shape of the melting curve is an intermediate shape between cis and trans forms, it may be determined that the cis and trans forms are mixed.

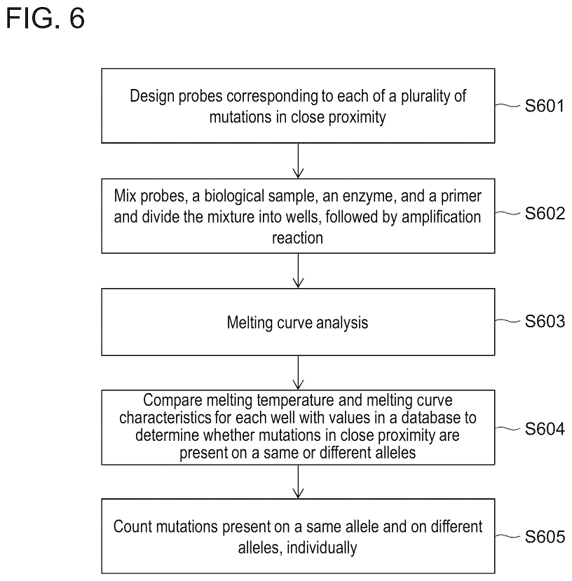

FIG. 6 is a flowchart showing one embodiment of a method for genetic analysis using melting curve analysis in combination with digital PCR to quantify the number of copies of genetic mutations and a ratio of cis to trans forms of the genetic mutations. First, probes corresponding to each of a plurality of mutations in close proximity may be designed (S601). The probes, a biological sample, an enzyme, and a primer, may be mixed and divided into wells, followed by PCR (S602). The melting curve of the probe corresponding to each mutation and the amplified product may be obtained by melting curve analysis (S603). For each well, the melting temperature and melting curve characteristics may be compared with values in the database to determine whether mutations in close proximity are present on a same allele or different alleles and whether the well contains only one or both mutations (S604). Finally, wells containing DNA with mutations present on the same allele, wells containing DNA with mutations present on different alleles, wells containing DNA with only one mutation, and wells containing neither mutation may be counted to calculate the initial concentration of each by Poisson correction (S605).

For example, a probe for T790M may be modified with the fluorescent dye FAM, and a probe for C797S may be modified with the fluorescent dye Cy5. Each probe may be mixed with a biological sample containing a nucleic acid of the target gene, an enzyme, and a primer, followed by division of the mixture into wells. After the PCR, melting curve analysis may be performed to obtain a melting curve and its differential curve for each of FAM and Cy5, and a melting temperature calculated from the differential curve. For each well, it may be compared with the melting temperature obtained in advance using the control nucleic acid stored in the same database to determine whether T790M and C797S are in a positional relationship of a cis form or a trans form. Even if T790M and C797S nucleic acids in a trans form are inserted into the same well one by one, they may not be erroneously determined as a cis form because the corresponding melting temperatures are obtained. The wells containing the cis-form nucleic acid, wells containing T790M and C797S nucleic acids in a trans form one by one, wells containing the T790M nucleic acid, wells containing the C797S nucleic acid, and wells containing neither mutation may be counted to calculate the initial concentration of each by Poisson correction.

### (2) Methods for Assessing Trait Associated with Plurality of Genetic mutations

As described above, the method of the present invention can determine whether a plurality of genetic mutations are present, and if present, whether they are in cis or trans form, and thus can be used to assess a trait associated with the genetic mutations, such as drug responsiveness.

Thus, in one aspect, the present invention provides a method of conducting genetic analysis on a test biological sample from a subject to assess a trait associated with a plurality of genetic mutations, the method comprising the steps of;
performing the method for genetic analysis using the test biological sample from a subject to determine whether the plurality of genetic mutations are on a same allele in a cis form or on different alleles in a trans form in the test biological sample from the subject; and
assessing whether the subject has the trait associated with the plurality of genetic mutations.

Examples of the traits associated with a plurality of genetic mutations may include susceptibility to a disease or disorder and drug responsiveness. For example, drug responsiveness in patients with lung cancer is different between EGFR genetic mutations T790M and C797S in a cis form and in a trans form, and all EGFR-TKIs may be effective in patients without genetic mutations. When mutations T790M and C797S are present in a trans form, the EGFR-TKIs gefitinib, erlotinib, afatinib, and dacomitinib may be effective in the protein with mutation C797S, while the EGFR-TKI osimertinib may be effective in the protein with mutation T790M. When the mutations T790M and C797S are present in a cis form, on the other hand, the above drugs may not be effective.

Furthermore, when the above trait is drug responsive, for example, an appropriate drug can be selected and administered to a subject after determining a plurality of genetic mutations in the subject. Therefore, in another aspect, the present invention provides a method for treating or preventing a disease in a subject, the method comprising the steps of: determining a plurality of genetic mutations associated with drug responsiveness using a biological sample derived from the subject; evaluating the drug responsiveness of the subject; and administering an appropriate drug to the subject. The mutations T790M and C797S in the EGFR gene are described in detail as examples. A biological sample derived from a patient with lung cancer, for example, blood may be prepared. As described above, the DNA in the biological sample may be determined for the presence of the mutations T790M and C797S in the EGFR gene, and if the genetic mutations are present in a cis or trans form. If the patient with lung cancer does not have the genetic mutations, at least one drug selected from all EGFR-TKIs may be administered. If the mutations T790M and C797S are present in a trans form, the patient may be administered with at least one drug selected from the EGFR-TKIs gefitinib, erlotinib, afatinib, and dacomitinib, and osimertinib. On the other hand, if the patient with lung cancer has the mutations T790M and C797S in a cis form, the EGFR-TKIs drugs may have low drug efficacy, and a treatment method or drug other than the above EGFR-TKIs drugs (e.g., brigatinib, which is an ALK tyrosine kinase inhibitor) needs to be selected. In this way, the patient can be administered and treated with drugs with optimal efficacy, and unnecessary drug administration can be avoided.

### (3) Kit for Genetic Analysis

The method of the present invention described above can be performed more easily and simply by using a kit including a plurality of fluorescent-labeled probes for melting curve analysis corresponding to at least a plurality of genetic mutations.

In one aspect, the invention provides a kit for genetic analysis, the kit comprising: a primer pair for amplifying a region comprising a plurality of genetic mutations, the primer pair comprising a forward primer and a reverse primer; and
a plurality of probes for melting curve analysis, each corresponding to the plurality of genetic mutations, which bind to a product amplified using the forward primer and the reverse primer,
wherein the probes have different melting curve characteristics or melting temperatures when the plurality of genetic mutations are present on a same allele and on different alleles.

The forward primer and reverse primer may be as described in the previous section. In one embodiment, the concentrations of the forward and reverse primers included in the kit of the present invention may be different, so that the concentration of either one is higher (due to asymmetric PCR). Preferably, the concentration of the primer that synthesizes the complementary strand of the probe for melting curve analysis may be higher.

The kits according to the present invention may further include other components necessary for carrying out the amplification reaction, such as DNA polymerases and substrates. The kit may also include instructions describing procedures and protocols for determining a plurality of genetic mutations.

When there are a plurality of sets of a plurality of genetic mutations to be analyzed, a plurality of primers or probes may be prepared based on the genetic mutations to be detected and added at the same time to perform an amplification reaction (e.g., asymmetric PCR) on a biological sample containing the DNA of a plurality of genetic mutations to be analyzed. The plurality of probes can be designed by changing the melting temperature with the DNA of each of the genetic mutations to be analyzed or by changing the type of fluorescent dye to determine, after the amplification reaction, the type of the genetic mutations to be analyzed contained in the solution from the color of the fluorescence and the melting temperature of the solution.

The kit for genetic analysis of the present invention may be useful for determining the presence or absence of a plurality of genetic mutations and for determining the positional relationship (a cis form or a trans form) of these genetic mutations on alleles.

The kit for genetic analysis of the present invention can also be used to assess a trait (e.g., susceptibility to a disease or disorder and drug responsiveness) associated with a plurality of genetic mutations.

### EXAMPLES

### Example 1

In this Example, EGFR gene with the T790M mutation only and EGFR gene with the C797S mutation only, and EGFR gene with these mutations in a cis form and in a trans form are amplified by real-time PCR and determined by melting curve analysis.

Genomic DNAs (final concentration: 133 molecules/μL) containing the T790M mutation only, the C797S mutation only, the mutations in a cis form and in a trans form in EGFR gene were prepared, and a forward primer (final concentration: 0.25 μM), a reverse primer (final concentration: 2.0 μM), a fluorescent-labeled probe corresponding to the T790M mutation (final concentration: 0.5 μM), a fluorescent-labeled probe corresponding to the C797S mutation (final concentration: 0.5 μM), a blocker for the wild-type corresponding to T790M (final concentration: 0.5 M), a blocker for the wild-type corresponding to C797S (final concentration: 0.5 μM), and 1× Master Mix (containing DNA polymerase and dNTPs) required for PCR were added to prepare a PCR reaction solution. At this time, the concentration of the primer pair was added to be asymmetric so that the complementary DNA strand of the fluorescent-labeled probe was over-amplified (concentration of the reverse primer added was set to be high). The sequences of the primers, probes, and blockers were as follows.
Forward primer: 5′-CCTCACCTCCACCGTGCA-3′ (SEQ ID NO: 1)
Reverse primer: 5′-TCTTTGTGTTCCCGGACATAGTC-3′ (SEQ ID NO: 2)
Fluorescent-labeled probe corresponding to T790M mutation: 5′-TCATCATGCAGCTCAT-3′ (SEQ ID NO: 3)
Fluorescent-labeled probe corresponding to C797S mutation: 5′-CTTCGGCAGCCTCCTG-3′ (SEQ ID NO: 4)
Blocker for the wild-type corresponding to T790M: 5′-GCTCATCACGCAGCT-3′ (SEQ ID NO: 5) (underlined bases represent special base LNAs)
Blocker for the wild-type corresponding to C797S: 5′-CTTCGGCTGCCTCCT-3′ (SEQ ID NO: 6) (underlined bases represent special base LNAs)

The forward and reverse primers correspond to nt 2348 to 2360 and nt 2398 to 2420 of the DNA sequence of human EGFR gene, respectively. Note that all of the fluorescent-labeled probes use EasyBeacon (PentaBase), which has hydrophobic special bases near both terminals, and free fluorescent-labeled probes are designed so that they form an intramolecular hydrophobic bond. The fluorescent-labeled probe corresponding to the T790M mutation has FAM as a fluorescent dye attached to the 5′-terminal and BHQ-1 as a quencher attached to the 3′-terminal, and the fluorescent-labeled probe corresponding to the C797S mutation has Cy5 as a fluorescent dye attached to the 5′-terminal and BHQ-2 as a quencher attached to the 3′-terminal. In addition, the blocker for the wild-type contains a special base LNA (locked nucleic acid) in the sequence, which is known to have an effect of increasing the Tm value, and a special base (terminator) attached to the 3′-terminal to inhibit an extension reaction.

The PCR reaction was performed at 95° C. for 20 seconds, followed by 60 cycles of 95° C. for 1 second, then 60° C. for 20 seconds. After the reaction, changes in the fluorescent intensity were observed with a real-time PCR device while heating from 50° C. to 95° C. to measure and analyze melting curves.

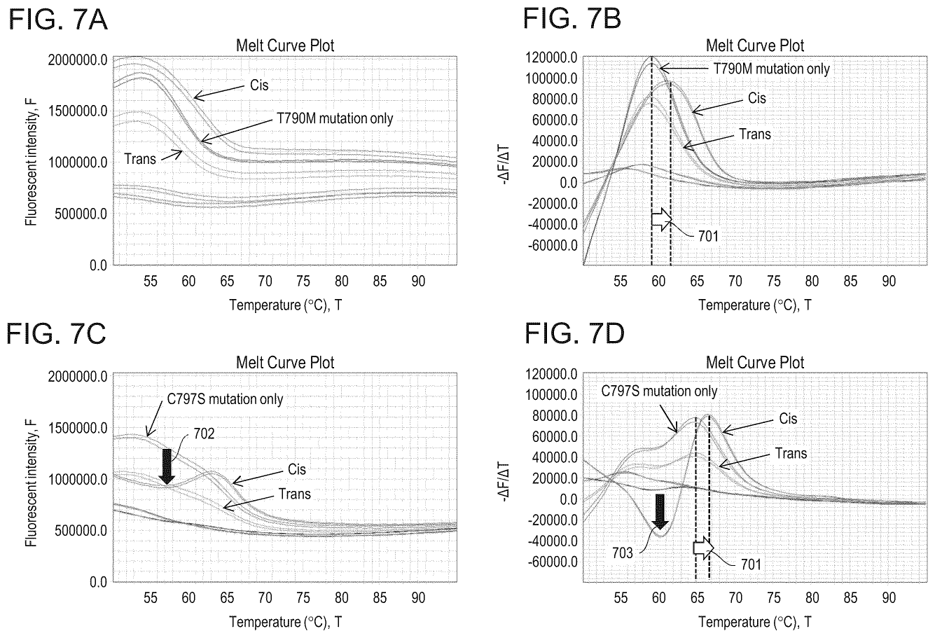

FIGS. 7A to 7D show melting curves measured with a FAM filter to observe the fluorescent dye FAM bound to the fluorescent-labeled probe corresponding to the T790M mutation. FIG. 7B shows differential curves of the melting curves in FIG. 7A. Comparing the melting temperatures, which are the peak tops of the differential curves of the melting curves of the respective genomic DNAs in FIG. 7B, it can be seen that the melting temperature of the genomic DNA of the cis-form (Cis) is 2 to 3° C. higher than that of the genomic DNA of the T790M mutation only and that of the genomic DNA of the trans-form (Trans) (shift in the melting temperatures indicated by an arrow 701).

FIG. 7C shows melting curves measured with a Cy5 filter to observe the fluorescent dye Cy5 bound to the fluorescent-labeled probe corresponding to the C797S mutation. Comparing the shapes of the melting curves of each genomic DNA, the genomic DNA of the C797S mutation only and the genomic DNA of trans-form (Trans) each had the shape of a sigmoid curve, whereas the genomic DNA of the cis-form (Cis) showed a dip in which the fluorescent intensity decreased and increased in a range of 50° C. to 65° C. The dip shape of the melting curve indicated by 702 is considered to be due to a reaction explained below. According to FIG. 7B, the melting temperature at which the fluorescent-labeled probe corresponding to the T790M mutation binds to the genomic DNA of the cis-form is 61° C. and according to FIG. 7A, the fluorescent intensity decreases in a high temperature range of 55° C. or higher, indicating that the binding amount of the probe decreases. Therefore, it is considered that the binding of the fluorescent-labeled probe corresponding to the C797S mutation to the genomic DNA of the cis-form was facilitated by the decreased binding of the fluorescent-labeled probe corresponding to the T790M mutation to the genomic DNA of the cis-form at 55° C. or higher, resulting in an increase in the fluorescence intensity at 55° C. or higher.

FIG. 7D shows differential curves of the melting curves measured with a Cy5 filter to observe the fluorescent dye Cy5 bound to the fluorescent-labeled probe corresponding to the C797S mutation shown in FIG. 7C. In the case of the C797S mutation, as well as the result of T790M in FIG. 7B, it can be seen that the melting temperature of the genomic DNA of the cis-form (Cis) is 2° C. higher than that of the genomic DNA of the C797S mutation only and that of the genomic DNA of the trans-form (Trans). In addition, the genomic DNA of the cis-form has a large negative peak around 60° C. in FIG. 7D, corresponding to the dip between 50° C. and 65° C. in FIG. 7C (negative peak at the lower temperature side than the melting temperature of the differential curve of the melting curve, indicated by an arrow 703). In the case of measurement of a sample in which the genomic DNA of the cis-form is mixed with the genomic DNA of the trans-form or the C797S mutation-only, the rate of shift in the melting temperature and the depth of the large negative peak around 60° C. in the differential curve of the melting curve are smaller than in the case of measurement of a sample with the genomic DNA of the cis-form alone. By assessing how much the genomic DNA of cis-form is shifted compared to the measurement of the sample with the genomic DNA of cis-form alone, it is possible to estimate a mixing rate of the genomic DNA of cis-form and the genomic DNA of trans-form or the C797S mutation-only.

Thus, the melting curve analysis can be conducted with fluorescent-labeled probes corresponding to each mutation in genetic mutations in close proximity to determine whether the genetic mutations are on a same allele (cis form) or on different alleles (trans form), whether only one mutation is present, or whether none of the mutations are present, based on the shape of the melting curve and the value of the melting temperature.

### Example 2

In this Example, EGFR gene with the T790M mutation only and EGFR gene with the C797S mutation only, and EGFR gene with these mutations in a cis form and in a trans form are amplified by digital PCR and determined by melting curve analysis.

Genomic DNAs (final concentration: 133 molecules/μL) containing the T790M mutation only, the C797S mutation only, the mutations in a cis form and in a trans form in EGFR gene were prepared, and a forward primer (final concentration: 0.25 μM), a reverse primer (final concentration: 2.0 μM), a fluorescent-labeled probe corresponding to the T790M mutation (final concentration: 0.5 μM), a fluorescent-labeled probe corresponding to the C797S mutation (final concentration: 0.5 μM), a blocker for the wild-type corresponding to T790M (final concentration: 0.5 μM), a blocker for the wild-type corresponding to C797S (final concentration: 0.5 μM), and 1× Master Mix (containing DNA polymerase and dNTPs) required for PCR were added to prepare a PCR reaction solution. At this time, the concentration of the primer pair was added to be asymmetric so that the complementary DNA strand of the fluorescent-labeled probe was over-amplified (concentration of the reverse primer added was set to be high). The primers, probes, and blockers were the same as those used in Example 1.

Subsequently, 15 μL of the PCR reaction solution was divided into 20,000 wells, and DNA was amplified by PCR. The PCR reaction was performed at 96° C. for 10 minutes, followed by 59 cycles of 60° C. for 2 minutes, then 98° C. for 30 seconds, and finally at 60° C. for 2 minutes. After the reaction, changes in the fluorescent intensity were observed in each well with a digital PCR device compatible with melting curve analysis while heating a chip provided with the wells from 50° C. to 85° C. on a temperature-controlled stage to measure and analyze melting curves.

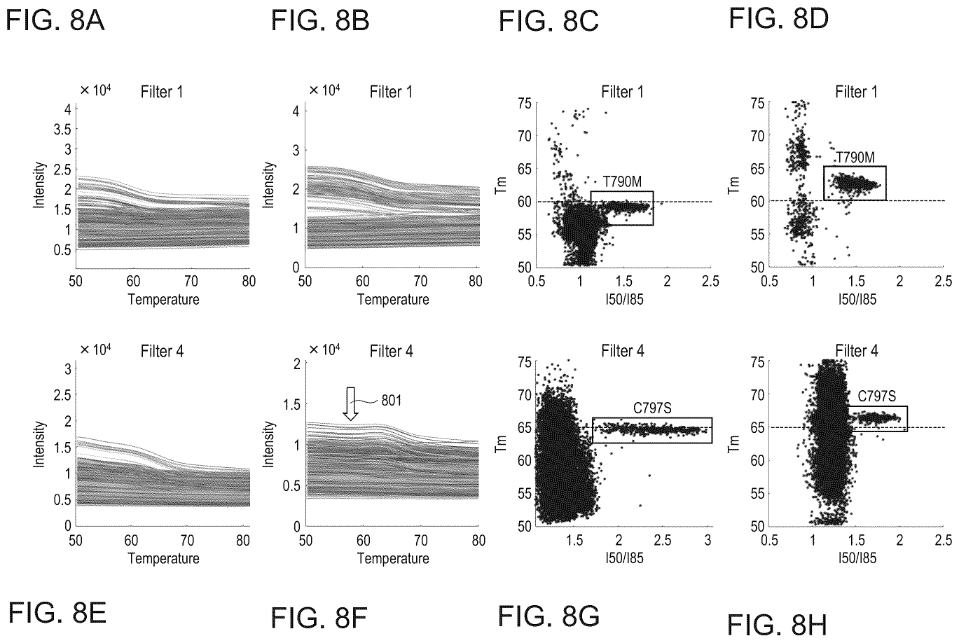

FIGS. 8A to 8H show melting curves obtained by digital PCR of the genomic DNA of trans-form and measured with a FAM filter to observe the fluorescent dye FAM bound to the fluorescent-labeled probe corresponding to the T790M mutation. FIG. 8B shows melting curves obtained by digital PCR of the genomic DNA of cis-form and measured with a FAM filter. FIG. 8C is a graph showing the relationship between the melting temperature and fluorescent intensity calculated from the melting curve in FIG. 8A. FIG. 8D is a graph showing the relationship between the melting temperature and fluorescent intensity calculated from the melting curve in FIG. 8B. Comparison of FIGS. 8C and 8D indicates that the melting temperature of the well containing the genomic DNA of cis-form is higher than that of the well containing the T790M mutation in the genomic DNA of trans-form.

FIG. 8E shows melting curves obtained by digital PCR of the genomic DNA of trans-form and measured with a Cy5 filter to observe the fluorescent dye Cy5 bound to the fluorescent-labeled probe corresponding to the C797S mutation. FIG. 8F shows melting curves obtained by digital PCR of the genomic DNA of cis-form and measured with a FAM filter. As shown in FIG. 8F, a dip 801 in the melting curve is observed in the well containing the genomic DNA of cis-form. FIG. 8G is a graph showing the relationship between the melting temperature and fluorescent intensity calculated from the melting curve in FIG. 8E. FIG. 8H is a graph showing the relationship between the melting temperature and fluorescent intensity calculated from the melting curve in FIG. 8F. Comparison of FIGS. 8G and 8H indicates that the melting temperature of the well containing the genomic DNA of cis-form is higher than that of the well containing the C797S mutation in the trans-form genomic DNA.

Thus, digital PCR may be performed using fluorescent-labeled probes corresponding to each mutation in genetic mutations in close proximity to conduct the melting curve analysis, thereby determining for each well whether the genetic mutations are on a same allele (cis form) or on different alleles (trans form), whether only one mutation is present, or whether none of the mutations are present, based on the shape of the melting curve and the value of the melting temperature. As a result, it is possible to count and quantify genes on a same allele (cis form), on different alleles (trans form), or only one mutation, based on the determination result for each well.

All publications, patents and patent applications cited in the present description are incorporated herein by reference in their entirety.

### DESCRIPTION OF SYMBOLS

101 DNA
102 Fluorescent-labeled probe
103 Fluorescent dye
104 Quencher
201 DNA containing T790M mutation in trans-form
202 DNA containing C797S mutation in trans-form
203 DNA containing T790M mutation and C797S mutation in cis-form
204 Fluorescent-labeled probe corresponding to T790M mutation
205 Fluorescent-labeled probe corresponding to C797S mutation
301 Melting temperature
302 Negative peak at lower temperature side than melting temperature
400 Database
701 Shift in melting temperature
702 Dip in melting curve
703 Negative peak at lower temperature side than melting temperature of the differential curve of the melting curve
801 Dip in melting curve

What is claimed is:

1. A method for genetic analysis comprising the steps of:
preparing probes corresponding to each of a plurality of genetic mutations;
performing an amplification reaction using a solution comprising a primer for amplifying a region comprising the plurality of genetic mutations, the probes, a test biological sample, and an enzyme;
measuring a binding of an amplicon to the probe by melting curve analysis at varying temperatures of the solution after performing the amplification reaction; and
determining, based on a result of the melting curve analysis, whether the plurality of genetic mutations are on a same allele in a cis form or on different alleles in a trans form in DNA contained in the test biological sample.

2. The method according to claim 1, wherein
the probe comprises a fluorescent dye or comprises a fluorescent dye and a quenching dye,
the fluorescent dye being used to measure the binding of the amplicon to the probe.

3. The method according to claim 1, wherein a 3′-terminal sequence and a 5′-terminal sequence of the probe have complementary sequence portion, and the probe forms a stem-loop structure in a free state.

4. The method of claim 1, wherein the step of performing the amplification reaction is conducted by an asymmetric polymerase chain reaction (PCR).

5. The method according to claim 1, comprising comparing a melting temperature for the DNA contained in the test biological sample using a first database that stores melting temperature information corresponding to each of cis and trans forms of the plurality of genetic mutations to determine whether the plurality of genetic mutations are in a cis form or in a trans form.

6. The method according to claim 5, comprising counting the number of copies of the genetic mutations, the number of copies of genetic mutations in a cis form, and the number of copies of genetic mutations in a trans form for the DNA contained in the test biological sample using the first database.

7. The method according to claim 1, comprising comparing a melting curve for the DNA contained in the test biological sample using a second database that stores melting curve characteristics corresponding to each of cis and trans forms of the plurality of genetic mutations to determine whether the plurality of genetic mutations are in a cis form or in a trans form.

8. The method according to claim 7, wherein the melting curve characteristics to be stored in the second database comprises at least one selected from the group consisting of a melting temperature, a left-right balance of a width of a peak shape in a differential curve of the melting curve, a magnitude of a local minimum on a low temperature side of a local maximum in a differential curve of the melting curve, a slope(s) of left and right sides of a peak in a differential curve of the melting curve and a balance thereof, a height of a local maximum and a height of a local minimum in a differential curve of the melting curve, and a ratio thereof.

9. The method according to claim 7, comprising counting the number of copies of the genetic mutations, the number of copies of genetic mutations in a cis form, and the number of copies of genetic mutations in a trans form for the DNA contained in the test biological sample using the second database.

10. The method according to claim 1, wherein in the step of performing the amplification reaction, an additional probe corresponding to a wild-type gene is added to the solution.

11. A method for conducting genetic analysis on a test biological sample from a subject to assess a trait associated with a plurality of genetic mutations, the method comprising the steps of:
performing the method according to claim 1 using the test biological sample from a subject to determine whether the plurality of genetic mutations are on a same allele in a cis form or on different alleles in a trans form in the test biological sample from the subject; and
assessing whether the subject has the trait associated with the plurality of genetic mutations.

12. The method according to claim 11, wherein the trait comprises susceptibility to a disease or disorder or drug responsiveness.

13. A kit for genetic analysis comprising:
a primer pair for amplifying a region comprising a plurality of genetic mutations, the primer pair comprising a forward primer and a reverse primer; and
a plurality of probes for melting curve analysis, each corresponding to the plurality of genetic mutations, which bind to a product amplified using the forward primer and the reverse primer,
wherein the probes have different melting curve characteristics or melting temperatures when the plurality of genetic mutations are present on a same allele and on different alleles.

14. The kit for genetic analysis according to claim 13, for carrying out a method comprising the steps of:
preparing probes corresponding to each of a plurality of genetic mutations;
performing an amplification reaction using a solution comprising a primer for amplifying a region comprising the plurality of genetic mutations, the probes, a test biological sample, and an enzyme;
measuring a binding of an amplicon to the probe by melting curve analysis at varying temperatures of the solution after performing the amplification reaction; and
determining, based on a result of the melting curve analysis, whether the plurality of genetic mutations are on a same allele in a cis form or on different alleles in a trans form in DNA contained in the test biological sample.


SEQUENCE LISTING (6 sequences)

SEQ ID NO: 1 - DNA, 18 bp, synthetic construct
  misc_feature 1..18: note = Primer
  source 1..18: mol_type = other DNA
cctcacctccaccgtgca

SEQ ID NO: 2 - DNA, 23 bp, synthetic construct
  misc_feature 1..23: note = Primer
  source 1..23: mol_type = other DNA
tctttgtgttcccggacatagtc

SEQ ID NO: 3 - DNA, 16 bp, synthetic construct
  misc_feature 1..16: note = Probe
  source 1..16: mol_type = other DNA
tcatcatgcagctcat

SEQ ID NO: 4 - DNA, 16 bp, synthetic construct
  misc_feature 1..16: note = Probe
  source 1..16: mol_type = other DNA
cttcggcagcctcctg

SEQ ID NO: 5 - DNA, 15 bp, synthetic construct
  misc_feature 1..15: note = Blocker sequence
  source 1..15: mol_type = other DNA
  modified_base 4: mod_base = OTHER; note = Locked Nucleic Acid (LNA)
  modified_base 8..10: mod_base = OTHER; note = Locked Nucleic Acid (LNA)
  modified_base 13..14: mod_base = OTHER; note = Locked Nucleic Acid (LNA)
gctcatcacgcagct

SEQ ID NO: 6 - DNA, 15 bp, synthetic construct
  misc_feature 1..15: note = Blocker sequence
  source 1..15: mol_type = other DNA
  modified_base 7..9: mod_base = OTHER; note = Locked Nucleic Acid (LNA)
cttcggctgcctcct